(19) United States
(12) Patent Application Publication
(10) Pub. No.: US 2013/0035533 A1
(43) Pub. Date: Feb. 7, 2013

(54) PROCESS FOR PURIFYING AROMATIC EXTRACTS CONTAINING AROMATIC POLYCYCLIC COMPOUNDS

(71) Applicants: Lotfi Hedhli, Noisy Le Roi (FR); Samuel Djelassi, La Chapelle Saint Luc (FR); Sylviane Pulvin, Compiegne (FR); Daniel Thomas, Villers Sur Coudun (FR)

(72) Inventors: Lotfi Hedhli, Noisy Le Roi (FR); Samuel Djelassi, La Chapelle Saint Luc (FR); Sylviane Pulvin, Compiegne (FR); Daniel Thomas, Villers Sur Coudun (FR)

(73) Assignee: TOTAL RAFFINAGE MARKETING, Puteaux (FR)

(21) Appl. No.: 13/638,178

(22) PCT Filed: Apr. 7, 2011
(86) PCT No.: PCT/IB2011/051503

§ 371 Date: Sep. 28, 2012

(30) Foreign Application Priority Data
Apr. 9, 2010 (FR) 1052733

(51) Int. Cl. C07C 7/152

(52) U.S. Cl. 585/804

(57) ABSTRACT

A process is disclosed for reducing the content of polycyclic aromatic hydrocarbons or PAHs in aromatic extracts including oxidizing the PAHs in the presence of a hemoprotein via an oxidizing compound, wherein the aromatic extract is brought into contact with the oxidizing agent in a non-reactive organic solvent, then is brought into contact with the immobilized or supported hemoprotein.

### CROSS-REFERENCE TO RELATED APPLICATIONS

This application is a National Phase Entry of International Application No. PCT/IB2011/051503, filed on Apr. 7, 2011, which claims priority to French Patent Application Ser. No. 1052733, filed on Apr. 9, 2010, both of which are incorporated by reference herein.

### BACKGROUND AND SUMMARY

The present invention relates to a method, also called process, for purifying aromatic extracts originating from the refining of crude oil containing highly toxic polynuclear aromatic hydrocarbons (PAHs), which are used as plasticizers in the industrial rubber and tyre industries.

The aromatic extracts are by-products of the method for obtaining lubricants in crude oil refining processes, in particular starting from products of the vacuum distillation of atmospheric residues. They result from single or double extraction of valuable raffinate from the lubricants, by means of a polar solvent, for example furfural, phenol or N-methylpyrrolidone or also from the hydrotreatment of these distillates and from a dewaxing stage. These extracts are identified at ETRMA (European Tyre and Rubber Manufacturers' Association) and correspond to the definition “complex combination of C20 to C50 hydrocarbons obtained by (1) solvent extraction from heavy petroleum distillates or (2) treatment of heavy petroleum distillates with hydrogen in the presence of a catalyst followed by dewaxing with solvent”. Until now, the DAEs or distillate aromatic extracts were the only ones to fully satisfy tyre industry specifications and were therefore indispensable. Because of the prohibition of the DAEs, other extracts are gradually being included in tyre formulations, such as MES or Mild Extract Solvent, or also TDAE or treated distillate aromatic extract. Certain extracts originate from deasphalting of vacuum residues followed by single or double extraction and are called, respectively, RAEs or Residual Aromatic Extracts and TRAEs or Treated Residual Aromatic Extracts.

The aromatic extract typically contains from 10 to 50% by weight of polycyclic aromatics (PCAs), 25 to 60% by weight of paraffinic compounds and 45 to 20% of naphthenic compounds. The aromatic extracts are widely used in the tyre and industrial rubber industries as they promote the reduction of energy losses which makes it possible to increase the production of these tyres or rubbers. Moreover, they improve the mechanical properties and low-temperature performances of the rubbers into which they are incorporated.

As a reminder, the PCA compounds are molecules made up of at least two rings of 5 to 6 carbon atoms, at least two carbon atoms of which are shared and at least one of the rings is aromatic. These PCA compounds differ by the number and the arrangement of the aromatic rings as well as by the substitutions of alkyl chains linked to the aromatic rings. Depending on the number of rings, the PCAs are classified as light PCA compounds (up to three rings) or heavy PCAs (more than three rings), and have very different physico-chemical and toxicological characteristics. Some of these polycyclic aromatic (PCA) compounds comprise at least one heterocycle with at least one sulphur, nitrogen and/or oxygen atom. Among these PCA compounds, some called PAHs or polynuclear aromatic hydrocarbons are classified in the list of major pollutants which are dangerous to public health due to their toxic character. Another feature is that unlike the PCAs, the PAHs do not comprise heterocycles in their chemical structure. These PAH compounds are biologically active molecules which can be absorbed by living organisms. Among these PAH compounds, benzo(a)pyrene (B(a)P), chrysene and benzo(b)fluoranthene are known to be particularly toxic. Depending on the chemical structure of the metabolites with which they are brought into contact and their mutagenic character, these toxic PAH compounds can result in a reduction in the response of the immune system thus increasing the risks of infection, or may even be carcinogenic.

In order to require the producers of aromatic extracts to reduce the content of toxic PAHs present in the aromatic extracts capable of having a negative influence on the environment, the European authorities published a new directive in 2005 (DIRECTIVE 2005/69/CE) which sets a limit of 1 ppm on the concentration of benzo(a)pyrene and of 10 ppm on all of the toxic PAHs cited in the directive, which corresponds to concentrations approximately ten times less than the current concentrations. Faced with this directive, the producers of aromatic extracts have attempted to reduce the content of toxic PAHs in the aromatic extracts or DAEs by different methods.

One of the first methods tested involved carrying out a second solvent extraction of the aromatic extract as described in the patent EP417980, typically with furfural. The product manufactured corresponds to TDAE. The method is very costly in terms of solvent, the yields are not always satisfactory, and the TDAE has a chemical composition different from that of DAEs and therefore very different physico-chemical properties.

Other methods, tested in order to remove the PAHs, consist of using bacteria such as Pseudomonas, Sphingomonas or Rhodococcus which have the ability to absorb PAHs with 2 and 3 rings such as naphthalene and phenanthrene. Some PAHs can be degraded by oxidation in the presence of lignolytic fungi such as Phanerochaete chrysosporium or microscopic fungi such as Cunninghamella elegans. However, these methods most often lead to the complete degradation of the aromatic molecules. The application of these methods to the aromatic extracts, apart from the prohibitive cost of these bacteria or fungi and the difficulty of utilizing them in industrial units, will result in compounds being obtained with properties very different from those of the aromatic extract currently incorporated in tyres and industrial rubbers. Another means is to carry out a partial opening of the aromatic rings by oxidizing them in a controlled fashion in order to form epoxidized and/or hydroxylated derivatives, for example aldehyde derivatives, quinones, acids and/or alcohols or also mixed compounds having lower toxicity with respect to the environment.

Thus the Applicant, in its patent FR2888246, describes the oxidation of the polycyclic aromatic compounds present in atmospheric distillation cuts of crude oil, in particular gasoline, kerosene and gas oil cuts, in the presence of an oxidant and of haemoprotein immobilized or not on a support. Like the aromatic extracts, these cuts contain polycyclic aromatic (PCA) compounds, but among the latter, this oxidation method is directed towards the degradation of compounds which are refractory to standard hydrogenation treatments, in particular benzothiophene and pyrrole derivatives. Due to the fluidity of the cuts and the low level of refractory aromatic compounds, this method takes place in an essentially aqueous medium, equally well in a reactive or non-reactive solvent, by bringing an oxidizing agent into contact with said cut to be treated in the presence of a haemoprotein immobilized or supported on divided solid particles. U.S. Pat. No. 6,461,859 describes a method of oxidation of the sulphur-containing compounds of petroleum cuts using a haemoprotein, in which the sulphur-containing PCAs are oxidized in a first stage then removed from the medium in a second distillation stage.

Within the context of the present invention, the Applicant is concerned with the degradation of the PAHs present in an aromatic extract, the properties of which are to be preserved and which are much more viscous and therefore more difficult to treat than the gas oil, gasoline and kerosene cuts. The viscosity of the aromatic extracts is such that it is not possible to identically apply the process used in the patent FR 2888246 directly to the aromatic extracts. In particular, bringing the haemoprotein into contact with the aromatic extract is subject to numerous pitfalls such as the use of a solvent aimed at reducing the viscosity of the aromatic extract which is essentially organic and which should not inhibit or slow down the oxidation reaction.

The present invention therefore relates to a method making it possible to degrade the PAHs in situ without modifying the mechanical and physical properties of the aromatic extract. Moreover, it relates to the implementation of an efficient method at relatively low cost. This degradation of the PAHs should be able to take place equally well continuously, discontinuously or semi-continuously depending on the quantity of aromatic extract to be treated. A subject of the present invention is therefore a method for reducing the content of polycyclic aromatic hydrocarbons or PAHs in the aromatic extracts which consists in oxidizing the PAHs in the presence of a haemoprotein using an oxidizing agent characterized in that the aromatic extract is brought into contact with the oxidizing agent in a non-reactive organic solvent, before being brought into contact with the immobilized or supported haemoprotein.

A first advantage of this method resides in the fact that the aromatic extract/oxidizing agent mixture is rendered homogeneous before being brought into contact with the catalyst of the reaction, here the supported or non-supported haemoprotein. This bringing into contact is facilitated by the reduction of the viscosity of the mixture by means of an appropriate solvent facilitating the homogenization of the mixture. Another advantage is that only the PAH compounds are reduced, the latter oxidizing first, and that all of the characteristics of these extracts remain unchanged.

In a particular embodiment of the present invention, the aromatic extract, solvent and oxidizing agent mixture is preferably homogenized before being brought into contact with the haemoprotein in order to promote the contacts and therefore the efficiency of the method. The temperature for bringing the aromatic extract, solvent and oxidizing agent mixture into contact with the haemoprotein varies from 15 to 80° C., preferably from 25 to 45° C. According to an additional embodiment, the method comprises a final stage of separation of the organic solvent from the aromatic extract treated, i.e. oxidized: the organic solvent thus recovered is advantageously recycled, a prior purification stage not being excluded.

The organic solvent is non-reactive, i.e. it does not react with the aromatic extract, oxidizing agent and haemoprotein mixture and does not form chemical, in particular covalent, bonds with the constituents of the mixture. Advantageously, the organic solvent is chosen from the group constituted by the dialkyl ketones, alkyl carboxylates, N-alkyl pyrrolidones and dimethyl sulphoxide or DMSO, capable of diluting the aromatic extracts. This solvent is preferably chosen from methyl ethyl ketone, acetone, ethyl ethanoate, methyl isobutyl ketone, ethyl acetate, N-methyl pyrrolidone (NMP). This choice is particularly important as the solvent must not denature the haemoprotein used or inhibit the oxidation reaction.

In this embodiment of the invention, the oxidizing agent is chosen from the oxidizing compounds soluble in organic medium. Among the latter, molecular oxygen (O₂), air, ozone (O₃), nascent hydrogen peroxide (H₂O₂), organic or mineral peroxides, alkylated hydroperoxides, aryl hydroperoxides and peracids are preferred. The invention is mainly applied to aromatic extracts comprising more than 10% of polycyclic aromatic compounds or PCAs, and preferably more than 20%. Moreover, these aromatic extracts comprise less than 70% by weight of a mixture of naphthenic and paraffinic compounds. In general, these aromatic extracts contain less than 500 ppm of PAH, preferably between 5 and 300 ppm. The aromatic extracts, the subject of the method according to the invention, preferably belong to the group constituted by the aromatic extracts of vacuum distillates such as DAE (distillate aromatic extract), MES and/or residual aromatic extracts or RAEs or also any extract resulting from an extraction of these aromatic extracts such as TDAE and/or TRAE.

In a preferred embodiment of the invention, the aromatic extract, solvent and oxidizing agent mixture is chosen in a respective concentration ratio (by weight) of these compounds varying from 40-10/90-60/0.001-2, and preferably varying from 30-20/80-70/0.1-1. More precisely, the method according to the invention comprises the following steps in the order specified:

i) a step of dissolution of 10-40% by weight of aromatic extract in an organic solvent,

ii) bringing the extract diluted in the solvent into contact with the oxidizing agent then the homogenization of the mixture,

iii) bringing the immobilized haemoprotein into contact with the homogenized mixture of step ii) by flushing or immersion,

iv) recovering then separating the treated extract from the solvent and, optionally,

v) recycling the solvent et step i), preferably after purification of the latter.

In order to implement the invention, the immobilized or supported haemoprotein is chosen from the haemoglobins and the myoglobins. It has also been noted that the method of immobilizing the haemoprotein is important in order to be efficient. Thus within the context of the present invention, the haemoprotein is immobilized on or in finely divided solid mineral particles of average size, determined by laser granulometry, comprised between 5 nm and 5 mm. Preferably, these particles have a size comprised between 10 nm and 2 mm. They are chosen from crystalline, amorphous or composite materials based on alkaline or alkaline-earth oxides, preferably from materials comprising alumina, silica, zirconia, titanium oxide or any composite material comprising at least one of these materials.

In order to immobilize the haemoprotein, the latter is absorbed on the surface of the solid particles and/or in the pores thereof in a ratio varying from 1 to 2000 mg of haemoprotein per g of mineral particles. The method of immobilizing the haemoprotein on these solid particles can be carried out in two ways; either by flushing a column filled with these solid particles with an aqueous solution of haemoprotein, for example varying from 5 to 100 g/l of haemoprotein, or by immersion of a haemoprotein in a water/acetone mixture, for example in a relative ratio of ⅓ by volume, the solid being dried before use. Among the haemoproteins envisaged, the haemoprotein, preferably chosen from the haemoglobins of bovine and/or pigs, is immobilized on divided silica with a particle size varying from 5 nm and 5 mm, in a content varying from 10 to 200 mg/g of silica. Reference can also be made to the Applicant's abovementioned application for more details relating to the haemoprotein, its preparation method, the support, the immobilization method, etc.

### BRIEF DESCRIPTION OF THE DRAWINGS

The invention will be described below with reference to the figures and examples given below.

FIG. 1 represents a diagram of the method according to the invention with continuous implementation of the method according to the invention.

FIG. 2 represents the % disappearance of the anthracene with furfural as solvent, as a function of time expressed in minutes in comparison with a control containing no oxidizing agent and for two oxidant/haemoprotein weight ratios of 400 and of 800 respectively.

FIG. 3 represents the % disappearance of the anthracene with methyl ethyl ketone as solvent, as a function of time expressed in minutes in comparison with a control containing no oxidizing agent and for two oxidant/haemoprotein weight ratios of 400 and of 800 respectively.

FIG. 4 represents the % disappearance of the anthracene with methyl ethyl ketone as solvent, as a function of time expressed in minutes.

### DETAILED DESCRIPTION

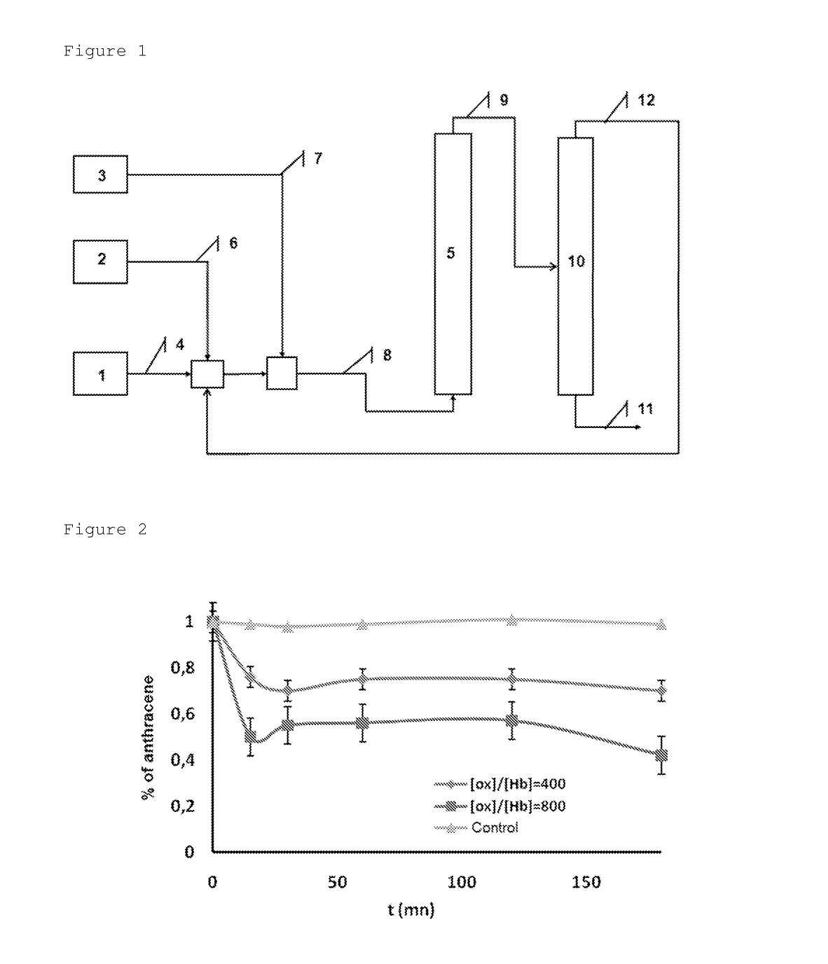

FIG. 1 shows three storage facilities for the aromatic extract, the solvent and the oxidant. The pipe sends the aromatic extract to the reactor containing the haemoprotein supported on solid particles. The pipes and successively convey the solvent making it possible to dilute the aromatic extract, then the oxidant, into the pipe the mixture being able to be homogenized in a method which is not shown. The homogenized mixture is introduced into the reactor via the pipe. The oxidized extract in a mixture in the solvent is removed via the pipe towards a separation facility for example a distillation column, the oxidized extract being recovered via the pipe and the solvent by the pipe. This pipe makes it possible to recycle the solvent optionally via a purification treatment which is not shown.

The examples given below aim at demonstrating the benefit of the invention without limiting its scope.

### Example 1

The aim of this example is to describe the preparation of a haemoprotein immobilized on divided solid particles by two preparation methods, the batch method for which the haemoprotein is absorbed into the solid particles and the continuous method consisting of flushing a bed of divided solid particles with a solution of haemoprotein. The batch method produces a haemoprotein X immobilized on silica, hereafter referred to as haemoprotein X.

1 g of Aerosil® 200 silica (Degussa), 200 mg of bovine haemoglobin (3.1 μmol) and 10 mL of phosphate buffer solution (pH 6, 50 mM) are introduced into a container suitable for a centrifuge. The mixture is stirred at 0° C. for 5 minutes, then 30 mL of acetone is added dropwise over a period of 10 minutes. Stirring of the mixture is continued for 30 minutes at 0° C., followed by centrifugation at 3000 rpm for 10 minutes. The solid residue collected in the bottom of the test tube is washed with 10 mL of phosphate buffer (pH 6) then centrifuged at 3000 rpm for 10 minutes, 3 times in succession. The resulting solid is then dried under vacuum in the presence of P₂O₅. The haemoprotein X contains 133 mg of haemoglobin per gram of silica. The continuous method produces a haemoprotein Y immobilized on silica, hereafter referred to as haemoprotein Y.

An aqueous solution containing 10 g/l of bovine haemoglobin is circulated at a rate of 1 ml/minute through a preparative HPLC column made of stainless steel 316 with an internal diameter of 10 mm and a length of 250 mm. This column is filled with 7.9 g of Silicagel®100 silica (Merck). The quantity of haemoglobin fixed on the silica is measured by the difference in the concentration of haemoglobin in the solution between the inlet and outlet of the column by UV-Vis spectrometric measurement at λ=404 nm. When the quantity of haemoglobin immobilized on the silica is sufficient, an aqueous solution to which an increasing concentration of acetone, from 100% of water to 1 to 5% of water, is circulated through the column, in order to carry away the excess non-absorbed haemoglobin and dry the haemoprotein Y. By this method, the quantity of haemoprotein adsorbed on the silica is approximately 90 mg/g of silica.

### Example 2

In the present example, the effect of the nature of the solvent on the degradation of anthracene, a model PAH molecule, is compared. The degradation of anthracene was compared in the presence of an oxidizing agent, either in solution in furfural or in solution with methyl ethyl ketone (MEK) under the same reaction conditions. Two mother liquids containing approximately 100 ppm of anthracene were therefore prepared in furfural and in MEK respectively. These solutions were used for preparing standard solutions for measuring the anthracene in the solutions degraded by means of HPLC-UV (λ=251.1 nm). 16 ml of solvent (furfural or MEK), then 4 ml of the mother liquid of anthracene (i.e. an anthracene concentration of 20 ppm in the mixture corresponding to the level of concentrations of the PAHs in an industrial solution) and finally, 800 mg of supported haemoprotein are introduced into 100 ml flasks placed under magnetic stirring. After homogenization of the mixture thus obtained, 20 and 40 μl of a solution of 70% tert-butyl peroxide in water is added, then the chronometer is started. Samples are regularly taken of each of the two mixtures containing furfural or MEK: they are diluted in acetonitrile then analyzed by HPLC.

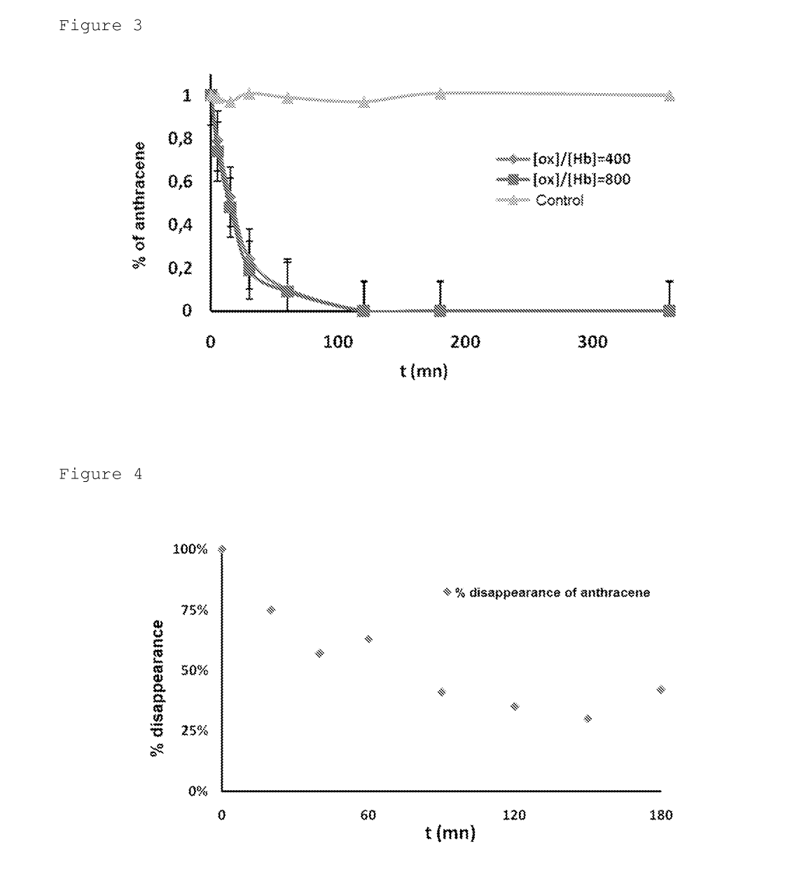

FIGS. 2 and 3 show the degradation of the anthracene resulting in a reduction in the concentration of anthracene, in the presence of an oxidant in the presence of furfural (FIG. 2) and of MEK (FIG. 3) respectively, in comparison with a control sample without oxidizing agent. It will be noted that the furfural inhibits the anthracene degradation reaction for both the oxidant/haemoglobin ratios; the furfural therefore gives lower results than the MEK.

### Example 3

In the present example, the continuous degradation of anthracene in the presence of a haemoprotein Y described in Example 1 is described. The column, filled with haemoprotein Y is continuously supplied at a flow rate of 1 ml/minute, with a solution of MEK containing 150 ppm of anthracene (ANT) and tert-butyl peroxide (tBuOH), the [tBuOH]/[ANT] molar ratio being fixed at 300. By comparing the anthracene contents measured between the inlet and the outlet of the column by GC-MS (coupling of gas chromatography/mass spectrometry), the quantity of degraded anthracene is determined. The graph corresponding to the disappearance of the anthracene as a function of time is given in FIG. 4.

### Example 4

In the present example, the degradation of PAHs in an aromatic extract by a continuous method in the presence of a haemoprotein Y as described in Example 3 is described. The aromatic extract is a DAE-type extract containing 15-25% of PCA including 1-0.01% of PAHs (TOTAL EXAROL 41). The content of several PAHs contained in the DAE between the inlet and the outlet of the column is measured as previously. The results are given in Table I below.

TABLE I

| | Concentration | Concentration | |
|---|---|---|---|
| | of PAHs | of PAHs | |
| | before column | after column | Degradation |
| | (ppm) | (ppm) | (%) |
| Benzo (a) anthracene | 6.5 | 5.4 | 18% |
| Chrysene | 26.4 | 23.5 | 11% |
| Benzo (b) fluoranthene | 16.3 | 12.8 | 21% |
| Benzo (k) fluoranthene | 5.5 | 4.2 | 23% |
| Benzo (a) pyrene | 18.5 | 13.3 | 28% |
| Dibenzo (a, h) | 17.4 | 11.5 | 34% |
| anthracene | | | |
| Benzo (b + j) | 16.3 | 12.8 | 21% |
| fluoranthene | | | |
| Benzo (e) pyrene | 64.6 | 42.7 | 34% |
| TOTAL | 171.5 | 126.2 | 26% |

The method according to the invention makes it possible to degrade more than 20% of the PAHs mentioned in the European Directive in most cases.

1. A method for reducing the content of polycyclic aromatic hydrocarbons or PAHs in aromatic extracts, the method comprising oxidizing the PAHs in the presence of a haemoprotein using an oxidizing agent, contacting the aromatic extract with the oxidizing agent in a non-reactive organic solvent and then contacting with the immobilized or supported haemoprotein.

2. The method according to claim 1 further comprising homogenization of the aromatic extract, solvent and oxidizing agent mixture before it is brought into contact with the haemoprotein.

3. The method according to claim 1, further comprising the temperature at which the aromatic extract, solvent and oxidizing agent mixture is brought into contact with the haemoprotein varies from 15 to 80° C.

4. The method according to claim 1, further comprising final step of separation of the treated aromatic extract from the organic solvent which is recycled.

5. The method according to claim 1, wherein the organic solvent is chosen from the group constituted by the dialkyl ketones, alkyl carboxylates, N-alkylpyrrolidones and dimethylsulphoxide or DMSO.

6. The method according to claim 1, wherein the organic solvent is chosen from the group constituted by methyl ethyl ketone, acetone, ethyl ethanoate, methyl isobutyl ketone, ethyl acetate, N-methylpyrrolidone (NMP).

7. The method according to claim 1, wherein the oxidizing agent is chosen from oxidizing compounds soluble in organic medium.

8. The method according to claim 1, wherein the oxidizing agent is chosen from molecular oxygen (O₂), air, ozone (O₃), nascent hydrogen peroxide (H₂O₂), organic or mineral peroxides, alkylated hydroperoxides, aryl hydroperoxides and peracids.

9. The method according to claim 1, wherein the aromatic extracts comprise more than 10% of polycyclic aromatic compounds or PCA.

10. The method according to claim 1, wherein the aromatic extracts comprise less than 70% by weight of a mixture of naphthenic and paraffinic compounds.

11. The method according to claim 1, wherein the aromatic extracts are chosen from the group constituted by the aromatic extracts of vacuum distillates, MES and/or residual aromatic extracts or RAEs or also any extract resulting from an extraction of these aromatic extracts such as TDAE and/or the TRAE.

12. The method according to claim 1, wherein the aromatic extract, solvent and oxidizing agent mixture corresponds to a respective weight ratio of these compounds varying from 40-10/90-60/0.001-2.

13. The method according to claim 1, wherein the aromatic extract, solvent and oxidizing agent mixture corresponds to a respective weight ratio of these compounds varying from 30-20/80-70/0.1-1.

14. The method according to claim 1, further comprising diluting the aromatic extract in the organic solvent before being mixed with the oxidizing agent, then homogenized.

15. A method for reducing the content of polycyclic aromatic hydrocarbons or PAHs in aromatic extracts, the method comprising:
(a) oxidizing the PAHs in the presence of a haemoprotein using an oxidizing agent, contacting the aromatic extract with the oxidizing agent in a non-reactive organic solvent and contacting with the immobilized or supported haemoprotein;
(b) dissolution of 10 to 40% by weight of aromatic extract in an organic solvent;
(c) bringing the extract diluted in the solvent into contact with the oxidizing agent, then homogenizing the mixture;
(d) bringing the immobilized haemoprotein into contact with the homogenized mixture of step (c) by flushing or immersion;
(e) recovering, then separating the treated extract from the solvent and, optionally; and
(f) recycling the solvent at step (b) after purification of the latter.

16. The method according to claim 1, wherein the immobilized or supported haemoprotein is chosen from the haemoglobins and the myoglobins.

17. The method according to claim 1, further comprising immobilizing the haemoprotein on or in finely divided solid mineral particles having an average size, determined by laser granulometry, comprised between 5 nm and 5 mm, these particles being chosen from the group of the crystalline, amorphous or composite materials based on alkaline or alkaline-earth oxides.

18. The method according to claim 1, further comprising absorbing the haemoprotein on the surface of the solid particles and/or in the pores thereof in a ratio varying from 1 to 2000 mg of haemoprotein per g of mineral particles.

19. The method according to claim 1, further comprising immobilizing the haemoprotein, which is a haemoglobin, on the solid particles and/or in the pores thereof in a ratio varying from 1 to 2000 mg of haemoprotein per g of mineral particles.

20. The method according to claim 17, wherein the particles are at least one of: alumina, silica, zirconia, titanium oxide or any composite material comprising at least one of these materials.
